(19) United States
(12) Patent Application Publication
(10) Pub. No.: US 2012/0029363 A1
(43) Pub. Date: Feb. 2, 2012

(54) SYSTEMS AND METHODS FOR IMPROVED COMPUTATION OF DIFFERENTIAL PULSE TRANSIT TIME FROM PHOTOPLETHYSMOGRAPH SIGNALS

(75) Inventor: Greg Lund, Boulder, CO (US)

(73) Assignee: Nellcor Puritan Bennett LLC, Boulder, CO (US)

(21) Appl. No.: 12/847,546

(22) Filed: Jul. 30, 2010

(51) Int. Cl. A61B 5/021

(52) U.S. Cl. 600/485

(57) ABSTRACT

Systems and methods for processing photoplethysmograph (PPG) signals to determine a differential pulse transit time (DPTT) are disclosed. Sensors may be used to obtain first and second PPG signals from a subject. The sensors may be placed at different locations on the subject's body. A first algorithm may be performed on the PPG signals or on signals derived from them to obtain a DPTT. A corresponding confidence measure may be determined and if the confidence measure falls within a first numerical range, the calculated DPTT may be used. On the other hand, if the confidence measure falls within a second numerical range, an alternative algorithm may be performed on the PPG signals or on signals derived from them and the DPTT obtained using the alternative algorithm may be used. The DPTT may be used to perform continuous or periodic measurements of blood pressure.

### SUMMARY

The present disclosure may relate to processing photoplethysmograph (PPG) signals and, more particularly, to systems and methods for computing a differential pulse transit time (DPTT) from a pair of PPG signals.

In an embodiment, probes or sensors may detect first and second PPG signals. The PPG signals may be detected at any suitable locations. For example, a first probe or sensor may detect a first PPG signal at a subject's earlobe, while a second probe or sensor may detect a second PPG signal at a subject's fingertip. These PPG signals may be processed to determine a DPTT, which may in turn be used to determine a blood pressure measurement.

In an embodiment, a maximum correlation algorithm may be performed based on the first and second PPG signals. A confidence measure may be determined using a first output generated by the maximum correlation algorithm. For example, the confidence measure may be indicative of how far a first derivative of the first PPG signal must be shifted in time relative to a first derivative of the second PPG signal in order to maximize correlation between the two first derivative waveforms. As another example, the confidence measure may be indicative of how highly correlated a first derivative of the first PPG signal is with a first derivative of the second PPG signal after one of the first derivative waveforms is shifted in time relative to the other. As another example, the confidence measure may be determined based on a combination of confidence measures.

In an embodiment, if the confidence measure is in a first numerical range, the DPTT may be determined based on the maximum correlation algorithm. In the example where the confidence measure may be indicative of a time shift that is required to maximize some measure of correlation based on the first and second PPG signals, the first numerical range may contain numbers greater than zero, corresponding to an indication that the first PPG signal (or a signal derived therefrom) must be shifted forward in time to maximize correlation with the second PPG signal (or a signal derived therefrom). If the first PPG signal corresponds to a measurement taken at a subject's earlobe and the second PPG signal corresponds to a measurement taken at a subject's fingertip, such a forward shift may be consistent with the observation that a given beat of the subject's heart usually produces a pulse at the subject's earlobe before producing a corresponding pulse at the subject's fingertip. In the example where the confidence measure may be indicative of how highly correlated the first PPG signal (or a signal derived therefrom) is with the second PPG signal (or a signal derived therefrom) after a time shift is performed, the first numerical range can include numbers greater than a certain threshold, corresponding to a relatively high degree of correlation between the PPG signals (or signals derived therefrom) after an appropriate time shift.

In an embodiment, if the confidence measure is in a second numerical range, an alternative algorithm may be performed based on the first and second PPG signals (or signals derived therefrom) and the DPTT may be determined based on the alternative algorithm. In the example where the confidence measure may be indicative of a time shift that is required to maximize some measure of correlation, the second numerical range may contain numbers less than zero, corresponding to an indication that the first PPG signal (or a signal derived therefrom) must be shifted backwards in time to maximize correlation with the second PPG signal (or a signal derived therefrom). If the first PPG signal corresponds to a measurement taken at a subject's earlobe and the second PPG signal corresponds to a measurement taken at a subject's fingertip, such a backward shift may be inconsistent with the observation that a given beat of the subject's heart usually produces a pulse at the subject's earlobe before producing a corresponding pulse at the subject's fingertip. In the example where the confidence measure may be indicative of how highly correlated the first PPG signal (or a signal derived therefrom) is with the second PPG signal (or a signal derived therefrom) after a time shift is performed, the second numerical range can include numbers less than a certain threshold, corresponding to a relatively low degree of correlation between the PPG signals (or signals derived therefrom) after an appropriate time shift.

In an embodiment, the alternative algorithm may be any suitable algorithm for determining a DPTT. For example, the alternative algorithm may include computing a second derivative of each of the first and second PPG signals, identifying a first set of peaks in the second derivative of the first PPG signal, identifying a second set of peaks in the second derivative of the second PPG signal, determining an average time difference between respective peaks in the first and second set of peaks, and outputting the average time difference as the DPTT. As another example, if the maximum correlation algorithm is performed using first derivatives of the first and second PPG signals, the alternative algorithm may include performing another maximum correlation algorithm based on the raw first and second PPG signals (without any derivative operations) or based on second derivatives of the first and second PPG signals. Any suitable DPTT algorithms may be used as the primary algorithm and the alternative algorithm.

In an embodiment, a system for processing first and second PPG signals to determine a DPTT may include a sensor (e.g., a pulse oximeter) capable of generating the PPG signal and a processor. The processor may be capable of receiving the first and second PPG signals, performing a maximum correlation algorithm based on the first and second PPG signal, and determining a confidence measure using a first output generated by the maximum correlation algorithm. The processor may further be capable of determining the DPTT based on the maximum correlation algorithm if the confidence measure is in a first numerical range. The processor may further be capable of performing an alternative algorithm based on the first and second PPG signals and determining the DPTT based on the alternative algorithm if the confidence measure is in a second numerical range different from the first numerical range.

In an embodiment, a computer-readable medium for processing first and second PPG signals to determine a DPTT may include computer program instructions. The computer program instructions recorded on the computer-readable medium may include instructions for receiving the first and second PPG signals, performing a maximum correlation algorithm based on the first and second PPG signal, and determining a confidence measure using a first output generated by the maximum correlation algorithm. The computer program instructions may further include instructions for determining the DPTT based on the maximum correlation algorithm if the confidence measure is in a first numerical range. The computer program instructions may further include instructions for performing an alternative algorithm based on the first and second PPG signals and determining the DPTT based on the alternative algorithm if the confidence measure is in a second numerical range different from the first numerical range.

### BRIEF DESCRIPTION OF THE DRAWINGS

The above and other features of the present disclosure, its nature and various advantages will be more apparent upon consideration of the following detailed description, taken in conjunction with the accompanying drawings in which:

FIG. 1 shows an illustrative CNIBP monitoring system in accordance with an embodiment;

FIG. 2 is a block diagram of the illustrative CNIBP monitoring system of FIG. 1 coupled to a patient in accordance with an embodiment;

FIG. 3 is a block diagram of an illustrative signal processing system in accordance with an embodiment;

FIG. 4 shows an illustrative PPG signal in accordance with an embodiment;

FIG. 5 shows an illustrative process for determining blood pressure from PPG signals in accordance with an embodiment;

FIG. 6 shows an illustrative process for determining a DPTT measurement from PPG signals in accordance with an embodiment;

FIG. 7 shows a first illustrative alternative algorithm for determining a DPTT measurement from PPG signals in accordance with an embodiment;

FIG. 8 shows a second illustrative alternative algorithm for determining a DPTT measurement from PPG signals in accordance with an embodiment;

FIG. 9 shows a set of illustrative waveforms depicting blood pressure determination using a DPTT measurement computed with a default maximum correlation algorithm in accordance with an embodiment; and

FIG. 10 shows a set of illustrative waveforms depicting blood pressure determination using a DPTT measurement computed with an alternative algorithm in accordance with an embodiment.

### DETAILED DESCRIPTION

Some CNIBP monitoring techniques may utilize two probes or sensors positioned at two different locations on a subject's body. The elapsed time, T, between the arrivals of corresponding points of a pulse signal at the two locations may then be determined using signals obtained by the two probes or sensors. The estimated blood pressure, p, may then be related to the elapsed time, T, by

p=a+b·ln(T) (1)

where a and b are constants that may be dependent upon the nature of the subject and the nature of the signal detecting devices. Other suitable equations using an elapsed time between corresponding points of a pulse signal may also be used to derive an estimated blood pressure measurement. In some embodiments, a single probe or sensor may be used, in which case the variable T in equation (1) would represent the time between two characteristic points within a single detected PPG signal. In still other embodiments, the area under at least part of a detected PPG signal may be used to compute blood pressure instead of time.

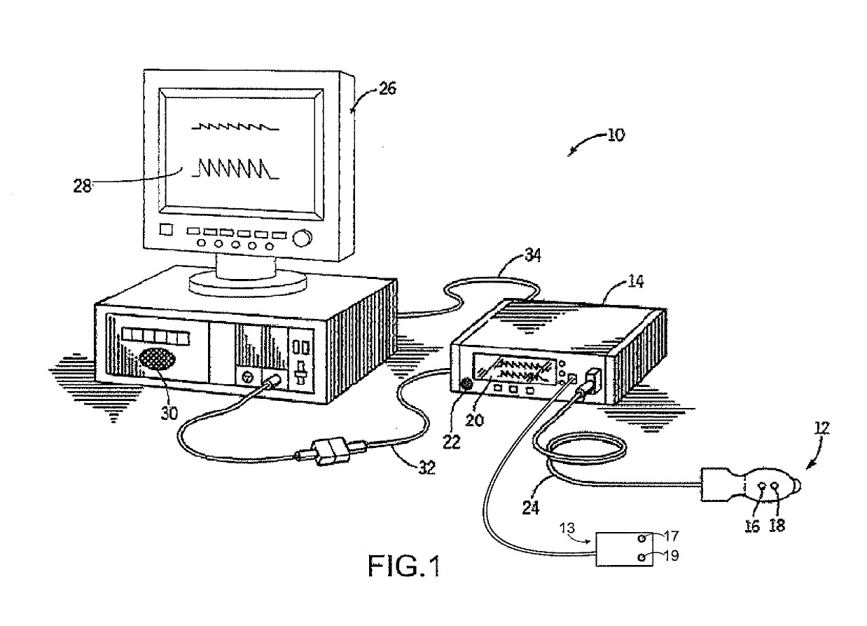

FIG. 1 is a perspective view of an embodiment of a CNIBP monitoring system 10 that may also be used to perform pulse oximetry. System 10 may include sensors 12 and 13 and a monitor 14. Sensor 12 may include an emitter 16 for emitting light at one or more wavelengths into a patient's tissue. A detector 18 may also be provided in sensor 12 for detecting the light originally from emitter 16 that emanates from the patient's tissue after passing through the tissue. Similarly, sensor 13 may include an emitter 17 and a detector 19, which may operate in a fashion similar to that of emitter 16 and detector 18, respectively.

Sensors 12 and 13 may be attached to different locations of a patient's body in order to measure values for time T in equation (1) above and thereby facilitate measurement of the patient's blood pressure. As an example, sensor 12 may be attached to the patient's fingertip, while sensor 13 may be attached to the patient's earlobe. It will be appreciated that other sensor locations may be used, as appropriate, and in some embodiments, only a single sensor or probe may be used.

According to an embodiment, emitter 16 and detector 18 may be on opposite sides of a digit such as a finger or toe, in which case the light that is emanating from the tissue has passed completely through the digit. In an embodiment, detector 18 (e.g., a reflective sensor) may be positioned anywhere a strong pulsatile flow may be detected (e.g., over arteries in the neck, wrist, thigh, ankle, ear, or any other suitable location). In an embodiment, emitter 16 and detector 18 may be arranged so that light from emitter 16 penetrates the tissue and is reflected by the tissue into detector 18, such as a sensor designed to obtain pulse oximetry or CNIBP data from a patient's forehead.

Similarly, according to an embodiment, emitter 17 and 19 may be on opposite sides of an ear (e.g., positioned on opposite sides of a patient's earlobe). In an embodiment, emitter 17 and detector 19 may be arranged so that light from emitter 17 penetrates the tissue and is reflected by the tissue into detector 19, such as a sensor designed to obtain pulse oximetry or CNIBP data from a patient's forehead.

According to another embodiment, system 10 may include a plurality of sensors forming a sensor array in lieu of either or both of sensors 12 and 13. Each of the sensors of the sensor array may be a complementary metal oxide semiconductor (CMOS) sensor. Alternatively, each sensor of the array may be charged coupled device (CCD) sensor. In another embodiment, the sensor array may be made up of a combination of CMOS and CCD sensors. The CCD sensor may comprise a photoactive region and a transmission region for receiving and transmitting data whereas the CMOS sensor may be made up of an integrated circuit having an array of pixel sensors. Each pixel may have a photodetector and an active amplifier.

In an embodiment, the sensors or sensor array may be connected to and draw its power from monitor 14 as shown. In another embodiment, the sensors may be wirelessly connected to monitor 14 and may each include its own battery or similar power supply (not shown). Monitor 14 may be configured to calculate physiological parameters (e.g., blood pressure) based at least in part on data received from sensors 12 and 13 relating to light emission and detection. In an alternative embodiment, the calculations may be performed on the monitoring device itself and the result of the light intensity reading may be passed to monitor 14. Further, monitor 14 may include a display 20 configured to display the physiological parameters or other information about the system. In the embodiment shown, monitor 14 may also include a speaker 22 to provide an audible sound that may be used in various other embodiments, such as for example, sounding an audible alarm in the event that a patient's physiological parameters are not within a predefined normal range.

In an embodiment, sensors 12 and 13 may be communicatively coupled to monitor 14 via cables 24 and 25, respectively. However, in other embodiments, a wireless transmission device (not shown) or the like may be used instead of or in addition to either or both of cables 24 and 25.

In the illustrated embodiment, system 10 may also include a multi-parameter patient monitor 26. The monitor may be cathode ray tube type, a flat panel display (as shown) such as a liquid crystal display (LCD) or a plasma display, or any other type of monitor now known or later developed. Multi-parameter patient monitor 26 may be configured to calculate physiological parameters and to provide a display 28 for information from monitor 14 and from other medical monitoring devices or systems (not shown). For example, multi-parameter patient monitor 26 may be configured to display an estimate of a patient's blood pressure from monitor 14, blood oxygen saturation generated by monitor 14 (referred to as an “SpO₂” measurement), and pulse rate information from monitor 14.

Monitor 14 may be communicatively coupled to multi-parameter patient monitor 26 via a cable 32 or 34 that is coupled to a sensor input port or a digital communications port, respectively and/or may communicate wirelessly (not shown). In addition, monitor 14 and/or multi-parameter patient monitor 26 may be coupled to a network to enable the sharing of information with servers or other workstations (not shown). Monitor 14 may be powered by a battery (not shown) or by a conventional power source such as a wall outlet.

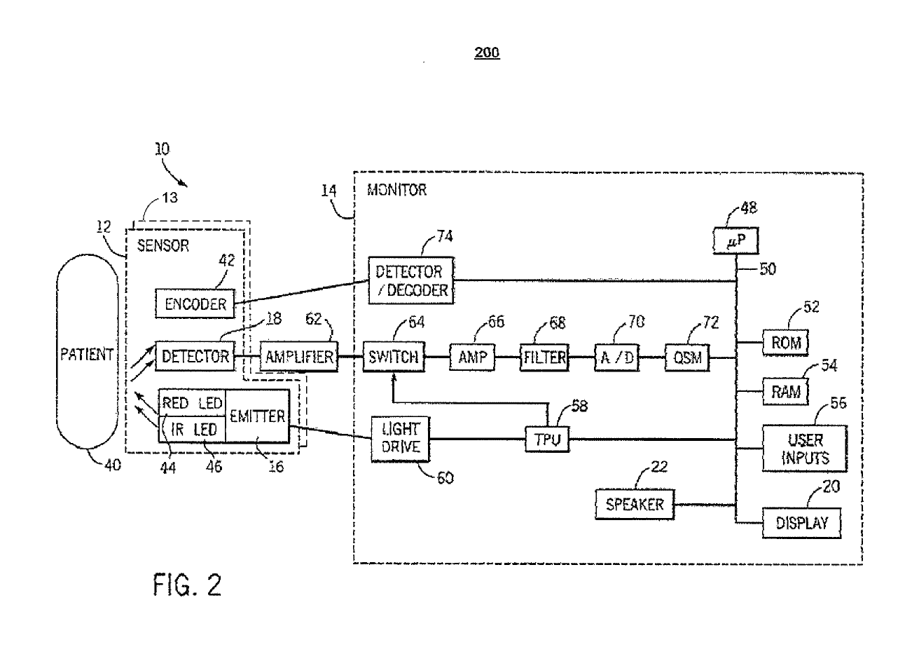

FIG. 2 is a block diagram of a CNIBP monitoring system, such as system 10 of FIG. 1, which may be coupled to a patient 40 in accordance with an embodiment. Certain illustrative components of sensors 12 and 13 and monitor 14 are illustrated in FIG. 2. Because sensors 12 and 13 may include similar components and functionality, only sensor 12 will be discussed in detail for ease of illustration. It will be understood that any of the concepts, components, and operation discussed in connection with sensor 12 may be applied to sensor 13 as well (e.g., emitter 16 and detector 18 of sensor 12 may be similar to emitter 17 and detector 19 of sensor 13). Similarly, it will be understood that, as discussed in connection with FIG. 1, certain embodiments may use only a single sensor or probe, instead of a plurality of sensors or probes as illustrated in FIG. 2.

Sensor 12 may include emitter 16, detector 18, and encoder 42. In the embodiment shown, emitter 16 may be configured to emit at least one wavelength of light (e.g., RED or IR) into a patient's tissue 40. For calculating SpO₂, emitter 16 may include a RED light emitting light source such as RED light emitting diode (LED) 44 and an IR light emitting light source such as IR LED 46 for emitting light into the patient's tissue 40. In other embodiments, emitter 16 may include a light emitting light source of a wavelength other than RED or IR. In one embodiment, the RED wavelength may be between about 600 nm and about 700 nm, and the IR wavelength may be between about 800 nm and about 1000 nm. In embodiments where a sensor array is used in place of single sensor, each sensor may be configured to emit a single wavelength. For example, a first sensor emits only a RED light while a second only emits an IR light.

It will be understood that, as used herein, the term “light” may refer to energy produced by radiative sources and may include one or more of ultrasound, radio, microwave, millimeter wave, infrared, visible, ultraviolet, gamma ray or X-ray electromagnetic radiation. As used herein, light may also include any wavelength within the radio, microwave, infrared, visible, ultraviolet, or X-ray spectra, and that any suitable wavelength of electromagnetic radiation may be appropriate for use with the present techniques. Detector 18 may be chosen to be specifically sensitive to the chosen targeted energy spectrum of the emitter 16.

In an embodiment, detector 18 may be configured to detect the intensity of light at the emitted wavelengths (or any other suitable wavelength). Alternatively, each sensor in the array may be configured to detect an intensity of a single wavelength. In operation, light may enter detector 18 after passing through the patient's tissue 40. Detector 18 may convert the intensity of the received light into an electrical signal. The light intensity is directly related to the absorbance and/or reflectance of light in the tissue 40. That is, when more light at a certain wavelength is absorbed or reflected, less light of that wavelength is received from the tissue by the detector 18. After converting the received light to an electrical signal, detector 18 may send the signal to monitor 14, where physiological parameters may be calculated based on the absorption of one or more of the RED and IR (or other suitable) wavelengths in the patient's tissue 40.

In an embodiment, encoder 42 may contain information about sensor 12, such as what type of sensor it is (e.g., whether the sensor is intended for placement on a forehead or digit) and the wavelength or wavelengths of light emitted by emitter 16. This information may be used by monitor 14 to select appropriate algorithms, lookup tables and/or calibration coefficients stored in monitor 14 for calculating the patient's physiological parameters.

Encoder 42 may contain information specific to patient 40, such as, for example, the patient's age, weight, and diagnosis. This information may allow monitor 14 to determine, for example, patient-specific threshold ranges in which the patient's physiological parameter measurements should fall and to enable or disable additional physiological parameter algorithms. Encoder 42 may, for instance, be a coded resistor which stores values corresponding to the type of sensor 12 or the type of each sensor in the sensor array, the wavelength or wavelengths of light emitted by emitter 16 on each sensor of the sensor array, and/or the patient's characteristics. In another embodiment, encoder 42 may include a memory on which one or more of the following information may be stored for communication to monitor 14: the type of the sensor 12; the wavelength or wavelengths of light emitted by emitter 16; the particular wavelength each sensor in the sensor array is monitoring; a signal threshold for each sensor in the sensor array; any other suitable information; or any combination thereof.

In an embodiment, signals from detector 18 and encoder 42 may be transmitted to monitor 14. In the embodiment shown, monitor 14 may include a general-purpose microprocessor 48 connected to an internal bus 50. Microprocessor 48 may be adapted to execute software, which may include an operating system and one or more applications, as part of performing the functions described herein. Also connected to bus 50 may be a read-only memory (ROM) 52, a random access memory (RAM) 54, user inputs 56, display 20, and speaker 22.

RAM 54 and ROM 52 are illustrated by way of example, and not limitation. Any suitable computer-readable media may be used in the system for data storage. Computer-readable media are capable of storing information that can be interpreted by microprocessor 48. This information may be data or may take the form of computer-executable instructions, such as software applications, that cause the microprocessor to perform certain functions and/or computer-implemented methods. Depending on the embodiment, such computer-readable media may include computer storage media and communication media. Computer storage media may include volatile and non-volatile, removable and non-removable media implemented in any method or technology for storage of information such as computer-readable instructions, data structures, program modules or other data. Computer storage media may include, but is not limited to, RAM, ROM, EPROM, EEPROM, flash memory or other solid state memory technology, CD-ROM, DVD, or other optical storage, magnetic cassettes, magnetic tape, magnetic disk storage or other magnetic storage devices, or any other medium which can be used to store the desired information and which can be accessed by components of the system.

In the embodiment shown, a time processing unit (TPU) 58 may provide timing control signals to a light drive circuitry 60, which may control when emitter 16 is illuminated and multiplexed timing for the RED LED 44 and the IR LED 46 for each sensor. TPU 58 may also control the gating-in of signals from detector 18 through an amplifier 62 and a switching circuit 64. These signals are sampled at the proper time, depending upon which light source is illuminated. The received signal from detector 18 may be passed through an amplifier 66, a low pass filter 68, and an analog-to-digital converter 70. The digital data may then be stored in a queued serial module (QSM) 72 (or buffer) for later downloading to RAM 54 as QSM 72 fills up. In one embodiment, there may be multiple separate parallel paths having amplifier 66, filter 68, and A/D converter 70 for multiple light wavelengths or spectra received.

In an embodiment, microprocessor 48 may determine the patient's physiological parameters, such as blood pressure, SpO₂, and pulse rate, using various algorithms and/or look-up tables based on the value of the received signals and/or data corresponding to the light received by detector 18. Signals corresponding to information about patient 40, and particularly about the intensity of light emanating from a patient's tissue over time, may be transmitted from encoder 42 to a decoder 74. These signals may include, for example, encoded information relating to patient characteristics. Decoder 74 may translate these signals to enable the microprocessor to determine the thresholds based on algorithms or look-up tables stored in ROM 52. User inputs 56 may be used to enter information about the patient, such as age, weight, height, diagnosis, medications, treatments, and so forth. In an embodiment, display 20 may exhibit a list of values which may generally apply to the patient, such as, for example, age ranges or medication families, which the user may select using user inputs 56.

The optical signal through the tissue can be degraded by noise, among other sources. One source of noise is ambient light that reaches the light detector. Another source of noise is electromagnetic coupling from other electronic instruments. Movement of the patient also introduces noise and affects the signal. For example, the contact between the detector and the skin, or the emitter and the skin, can be temporarily disrupted when movement causes either to move away from the skin. In addition, because blood is a fluid, it responds differently than the surrounding tissue to inertial effects, thus resulting in momentary changes in volume at the point to which the sensor or probe is attached.

Noise (e.g., from patient movement) can degrade a CNIBP or pulse oximetry signal relied upon by a physician, without the physician's awareness. This is especially true if the monitoring of the patient is remote, the motion is too small to be observed, or the doctor is watching the instrument or other parts of the patient, and not the sensor site. Processing CNIBP or pulse oximetry (i.e., PPG) signals may involve operations that reduce the amount of noise present in the signals or otherwise identify noise components in order to prevent them from affecting measurements of physiological parameters derived from the PPG signals.

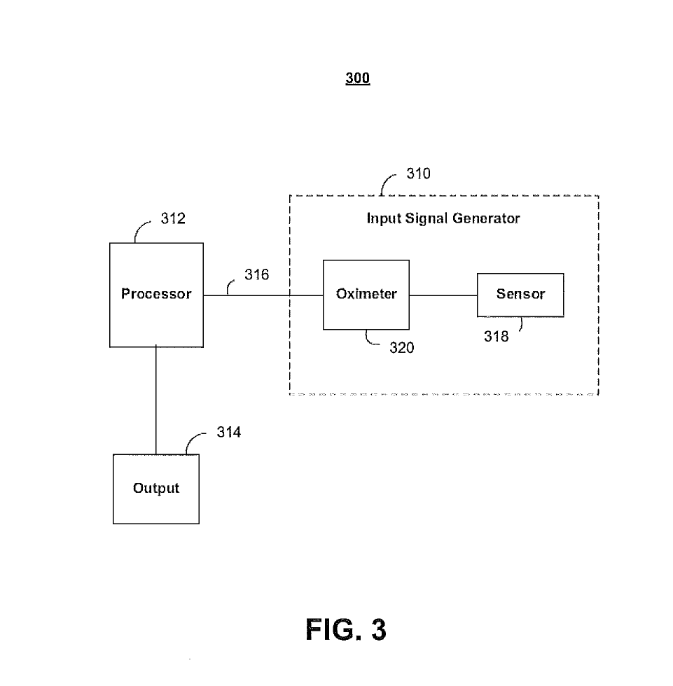

FIG. 3 is an illustrative processing system 300 in accordance with an embodiment. In this embodiment, input signal generator 310 generates an input signal 316. As illustrated, input signal generator 310 may include oximeter 320 (or similar device) coupled to sensor 318, which may provide as input signal 316 a PPG signal. It will be understood that input signal generator 310 may include any suitable signal source, signal generating data, signal generating equipment, or any combination thereof to produce signal 316. Additionally, input signal generator 310 may in some embodiments include more than one sensor 318.

In this embodiment, signal 316 may be coupled to processor 312. Processor 312 may be any suitable software, firmware, and/or hardware, and/or combinations thereof for processing signal 316. For example, processor 312 may include one or more hardware processors (e.g., integrated circuits), one or more software modules, computer-readable media such as memory, firmware, or any combination thereof. Processor 312 may, for example, be a computer or may be one or more chips (i.e., integrated circuits). Processor 312 may perform some or all of the calculations associated with the blood pressure monitoring methods of the present disclosure. For example, processor 312 may determine the time difference, T, between any two chosen characteristic points of a PPG signal obtained from input signal generator 310. As another example, if input signal generator contains more than one sensor 318, processor 312 may determine the time difference, T, required for a PPG signal to travel from one sensor 318 to another. Processor 312 may also be configured to apply equation (1) (or any other blood pressure equation using an elapsed time value) and compute estimated blood pressure measurements on a continuous or periodic basis. Processor 312 may also perform any suitable signal processing of signal 316 to filter signal 316, such as any suitable band-pass filtering, adaptive filtering, closed-loop filtering, and/or any other suitable filtering, and/or any combination thereof. For example, signal 316 may be filtered one or more times prior to or after identifying characteristic points in signal 316.

Processor 312 may be coupled to one or more memory devices (not shown) or incorporate one or more memory devices such as any suitable volatile memory device (e.g., RAM, registers, etc.), non-volatile memory device (e.g., ROM, EPROM, magnetic storage device, optical storage device, flash memory, etc.), or both. Processor 312 may perform initial calibration, recalibration, or both of the CNIBP measuring system, using information received from input signal generator 310 or any other suitable device.

Processor 312 may be coupled to output 314. Output 314 may be any suitable output device such as, for example, one or more medical devices (e.g., a medical monitor that displays various physiological parameters, a medical alarm, or any other suitable medical device that either displays physiological parameters or uses the output of processor 212 as an input), one or more display devices (e.g., monitor, PDA, mobile phone, any other suitable display device, or any combination thereof), one or more audio devices, one or more memory devices (e.g., hard disk drive, flash memory, RAM, optical disk, any other suitable memory device, or any combination thereof), one or more printing devices, any other suitable output device, or any combination thereof.

It will be understood that system 300 may be incorporated into system 10 (FIGS. 1 and 2) in which, for example, input signal generator 310 may be implemented as parts of sensor 12 and monitor 14 and processor 312 may be implemented as part of monitor 14. In some embodiments, portions of system 300 may be configured to be portable. For example, all or a part of system 300 may be embedded in a small, compact object carried with or attached to the patient (e.g., a watch (or other piece of jewelry) or cellular telephone). In such embodiments, a wireless transceiver (not shown) may also be included in system 300 to enable wireless communication with other components of system 10. As such, system 10 may be part of a fully portable and continuous blood pressure monitoring solution.

As mentioned above, multi-parameter equation (1) may be used to determine estimated blood pressure measurements from the time difference, T, between two or more characteristic points of a PPG signal. In an embodiment, the PPG signals used in the CNIBP monitoring techniques described herein are generated by a pulse oximeter or similar device. Systems 10 (FIGS. 1 and 2) and 300 (FIG. 3) may also include a calibration device (e.g., an aneroid or mercury sphygmomanometer and occluding cuff) that generates blood pressure or other measurements to calibrate the CNIBP calculations.

The present disclosure may be applied to measuring systolic blood pressure, diastolic blood pressure, mean arterial pressure (MAP), or any combination of the foregoing on an on-going, continuous, or periodic basis. U.S. patent application Ser. No. 12/242,238 filed Sep. 30, 2008, which is hereby incorporated by reference herein in its entirety, discloses some techniques for continuous and non-invasive blood pressure monitoring that may be used in conjunction with the present disclosure.

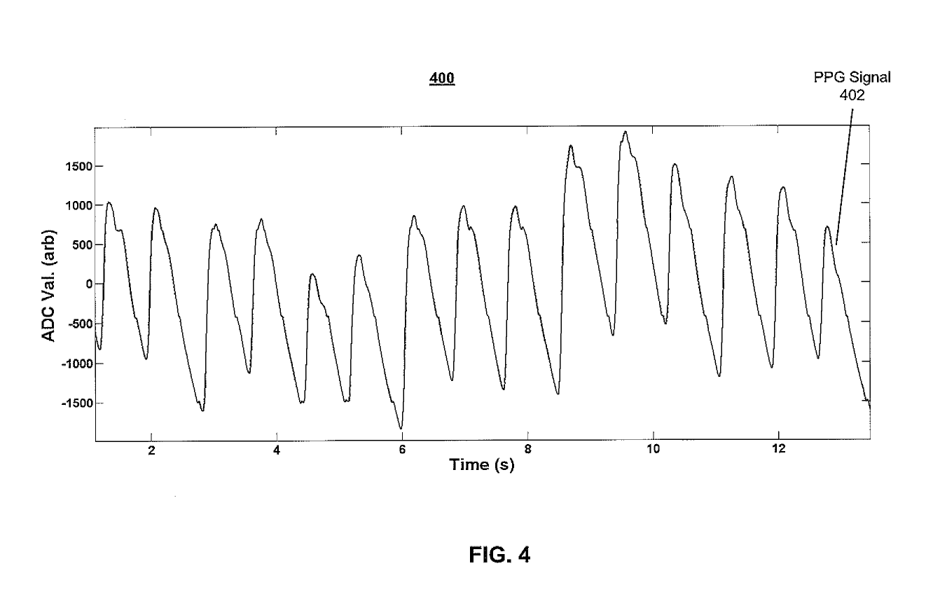

FIG. 4 shows illustrative PPG signal 400. As described above, in some embodiments PPG signal 400 may be generated by a pulse oximeter or similar device positioned at any suitable location of a subject's body. Additionally, PPG signal 400 may be generated at each of a plurality of locations of a subject's body, with at least one probe or sensor attached to each location. The time difference T that it takes for PPG signal 400 to appear at one location and another location (e.g., at a patient's ear and at the patient's finger or toe) may then be measured and used to derive a blood pressure measurement for the patient using a calibrated version of equation (1) or using any other relationship, such as lookup tables and the like. Time T may be measured, for example, by determining the difference between how long it takes for a given characteristic point, observed in the PPG signal at the first sensor or probe location, to appear in the PPG signal at the second sensor or probe location.

In an embodiment, PPG signal 400 may be generated using only a single sensor or probe attached to the subject's body. In such a scenario, the time difference, T, may correspond to the time it takes the pulse wave to travel a predetermined distance (e.g., a distance from the sensor or probe to a reflection point and back to the sensor or probe). Characteristic points in the PPG signal may include the time between various peaks in the PPG signal and/or in some derivative of the PPG signal. For example, in some embodiments, the time difference, T, may be calculated between (1) the maximum peak of the PPG signal in the time domain and the second peak in the 2nd derivative of the PPG signal (the first 2nd derivative peak may be close to the maximum peak in the time domain) and/or (2) peaks in the 2nd derivative of the PPG signal. Any other suitable time difference between any suitable characteristic points in the PPG signal (e.g., PPG signal 400) or any derivative of the PPG signal may be used as T in other embodiments.

In an embodiment, the time difference between the adjacent peaks in the PPG signal, the time difference between the adjacent valleys in the PPG signal, or the time difference between any combination of peaks and valleys, can be used as the time difference T. As such, adjacent peaks and/or adjacent valleys in the PPG signal (or in any derivative thereof) may also be considered characteristic points. In an embodiment, these time differences may be divided by the actual or estimated heart rate to normalize the time differences. In an embodiment, the resulting time difference values between two peaks may be used to determine the systolic blood pressure, and the resulting time difference values between two valleys may be used to determine the diastolic blood pressure.

Characteristic points in a PPG signal (e.g., PPG signal 400) may be identified in a number of ways. For example, in some embodiments, the turning points of 1st, 2nd, 3rd (or any other) derivative of the PPG signal are used as characteristic points. Additionally or alternatively, points of inflection in the PPG signal (or any suitable derivative thereof) may also be used as characteristic points of the PPG signal.

In an embodiment, blood pressure may be determined by, for example, measuring the area under a pulse or a portion of the pulse in the PPG signal (e.g., PPG signal 400). These measurements may be correlated with empirical blood pressure data (corresponding to previous blood pressure measurements of the patient or one or more other patients) to determine the blood pressure. In some implementations, the blood pressure may be determined by looking up the area measurement values in a table, which may be stored in a memory, to obtain corresponding blood pressures. Alternatively, the blood pressure may be determined by using any suitable blood pressure-area mapping equation which is generated based on blood pressure and area measurements associated with one or more patients. For example, measured samples may be plotted in a graph that maps blood pressure to area. The graph may be analyzed to generate a linear-best-fit-line approximation, non-linear best fit line approximation or other suitable approximation from which to derive an equation that may be used to determine blood pressure by providing an area measurement.

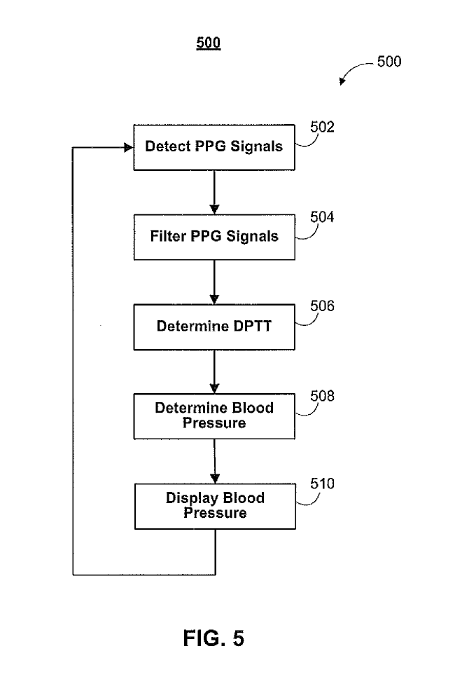

FIG. 5 shows an illustrative process 500 for determining blood pressure from PPG signals in accordance with an embodiment. At step 502, PPG signals may be detected from a patient. For example, monitor 14 (FIGS. 1 and 2) may be used to detect PPG signals from patient 40 (FIG. 2) using, for example, sensors such as sensors 12 and 13 (FIGS. 1 and 2). The sensors may be located at any suitable site on the patient, e.g., forehead, earlobe, toe, finger, or chest. In an embodiment, a first PPG signal may be detected from a sensor located relatively close to the patient's heart (e.g., the earlobe), while a second PPG signal may be detected from a sensor located relatively far from the patient's heart (e.g., the fingertip). The PPG signals may be detected by microprocessor 48 (FIG. 2) and/or processor 312 (FIG. 3) of a CNIBP monitoring or pulse oximetry system.

At step 504, the PPG signals detected at step 502 may be filtered using any suitable circuitry, such as filter 68 (FIG. 2), microprocessor 48 (FIG. 2), and/or processor 312 (FIG. 3) of CNIBP monitoring or pulse oximetry system 10 (FIG. 1) of a CNIBP monitoring or pulse oximetry system. Step 504 may include high-pass filtering, low-pass filtering, band-pass filtering, or any suitable combination thereof. For example, in an embodiment step 504 may include low-pass filtering the PPG signals detected at step 502, to eliminate relatively high-frequency noise, then high-pass filtering the signal that results from the low-pass filtering.

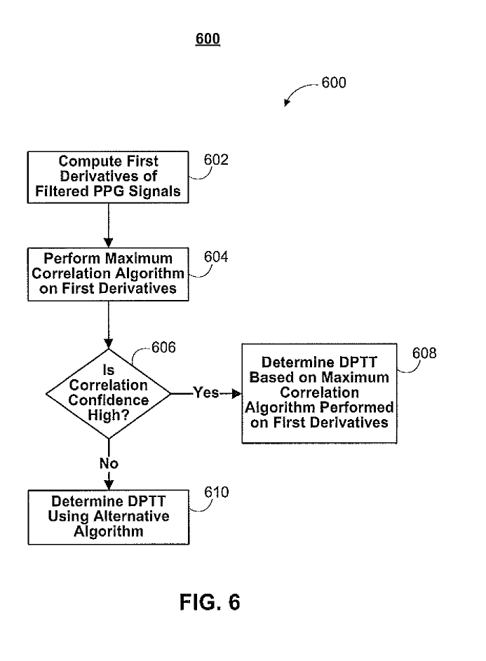

At step 506, a DPTT may be determined from the filtered PPG signals resulting from step 504. The DPTT determination at step 506 may be performed by microprocessor 48 (FIG. 2) and/or processor 312 (FIG. 3) of a CNIBP monitoring or pulse oximetry system, and may use any suitable algorithm, such as a correlation algorithm, a peak-picking algorithm, or any suitable combination thereof. In an embodiment, the DPTT determination in step 506 may be performed using process 600 (FIG. 6).

At step 508, a blood pressure measurement may be determined based, at least in part, on the DPTT determined at step 506. For example, equation (1) above (or any other blood pressure equation using an elapsed time between the arrival of corresponding points of a pulse signal or any other suitable computed time difference) may be used to compute estimated blood pressure measurements. In an embodiment, the computed time difference between characteristic points in a single PPG signal may be substituted for the elapsed time between the arrival of corresponding points of a pulse signal.

After blood pressure measurements are determined, the measurements may be outputted, stored, or displayed in any suitable fashion at step 510. For example, multi-parameter patient monitor 26 (FIG. 1) may display a patient's blood pressure on display 28 (FIG. 1). Additionally or alternatively, the measurements may be saved to memory or a storage device (e.g., ROM 52 or RAM 54 of monitor 14 (FIG. 2)) for later analysis or as a log of a patient's medical history.

In practice, one or more steps shown in process 500 may be combined with other steps, performed in any suitable order, performed in parallel (e.g., simultaneously or substantially simultaneously), or removed.

FIG. 6 shows an illustrative process 600 for determining a DPTT measurement from PPG signals in accordance with an embodiment. Process 600 may be used as part of step 506 of process 500 (FIG. 5). At step 602, the first derivative of each of the filtered PPG signals (e.g., generated at step 504 of FIG. 5) may be computed. The derivatives may be computed by microprocessor 48 (FIG. 2) and/or processor 312 (FIG. 3) of a CNIBP monitoring or pulse oximetry system. The use of first derivative waveforms may advantageously facilitate computation of a DPTT using systolic measurements. In another embodiment, a second derivative of the filtered PPG signals may be taken instead (e.g., to facilitate processing of a DPTT using diastolic measurements) or the filtered PPG signals themselves may be used without any derivative operations.

At step 604, a maximum correlation algorithm may be performed on the first derivatives computed at step 602. The maximum correlation algorithm may be computed by microprocessor 48 (FIG. 2) and/or processor 312 (FIG. 3) of a CNIBP monitoring or pulse oximetry system. By way of example, consider a scenario where one first derivative waveform computed at step 602 corresponds to a PPG signal measured at a patient's earlobe, while another first derivative waveform computed at step 602 corresponds to a PPG signal measured at the patient's fingertip. Each beat of the patient's heart typically results in the arrival of a pulse at the patient's earlobe before the arrival of a pulse at the patient's fingertip. Thus, a DPTT may be computed by determining how much time elapses between the arrival of a pulse at the earlobe and the arrival of a corresponding pulse at the fingertip. The maximum correlation algorithm may shift the two first-derivative waveforms relative to each other in time, and identify what amount of time shift results in the highest correlation between the two first-derivative waveforms. The correlation between the two waveforms may be measured based on peaks or valleys in the first-derivative waveforms, based on other relevant portions of the waveforms, or based on the waveforms in their entirety. The amount of time shift resulting in the highest correlation may then be used as a DPTT measurement for the purpose of measuring a patient's blood pressure.

At step 606, a degree of confidence in the results of the maximum correlation algorithm of step 604 may be determined. The confidence determination may be computed by microprocessor 48 (FIG. 2) and/or processor 312 (FIG. 3) of a CNIBP monitoring or pulse oximetry system. Confidence may be determined by examining the DPTT generated at step 604, a measure of the degree of correlation resulting from the alignment chosen at step 604, or any other suitable metric or any combination thereof.

With respect to the time-based confidence determination, the maximum correlation algorithm is generally expected to yield a DPTT within a certain range. In the example described in connection with step 604, for instance, the difference between the time it takes for a pulse of blood to reach a patient's fingertip and the time for it to reach the patient's earlobe typically falls into a certain numerical range. Thus, a relatively low DPTT (e.g., falling below a certain threshold) may indicate a relatively low degree of confidence in the results of the maximum correlation algorithm. As an extreme example, a DPTT falling below zero (often referred to as a “zero crossing”) is typically an indication that the results of the maximum correlation algorithm are erroneous, as it is almost physiologically impossible for a given pulse of blood to arrive at the fingertip before it arrives at the ear. Likewise, a DPTT that is above a certain threshold may indicate a relatively high degree of confidence in the results of the maximum correlation algorithm performed in step 604. It will be noted that this discussion assumes that the first PPG signal corresponds to a location closer to the heart than the location used to measure the second PPG signal. If the first PPG signal is measured at a more remote location, then the DPTT determined at step 604 would be expected to be below a certain negative threshold.

With respect to the correlation-based confidence determination, maximum correlation algorithms may choose the DPTT based on the amount of time shift that maximizes some metric representing the amount of correlation between the first and second waveforms. The resulting maximum value of the correlation metric may then be examined as an indication of the degree of confidence in the DPTT computed. A relatively high correlation metric measurement (e.g., above a given threshold) may be indicative of a relatively high degree of confidence, while a relatively low correlation measurement (e.g., below a given threshold) may be indicative of a relatively low degree of confidence.

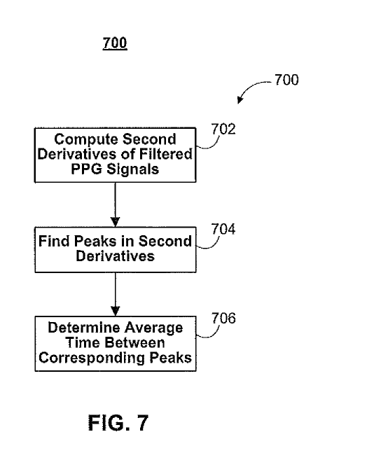

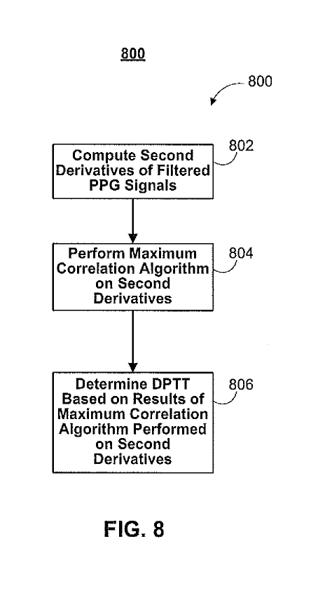

Thus, step 606 may determine whether or not the system is relatively confident in the results of the maximum correlation algorithm performed in step 604 using a time-based metric, a correlation based metric, any other suitable metric, or any combination thereof (e.g., a weighted sum). If a relatively high degree of confidence is detected at step 606, process 600 may proceed to step 608, where the DPTT may be set to the maximum-correlation time shift amount computed by the maximum correlation algorithm at step 604. On the other hand, if a relatively low degree of confidence is detected at step 606, process 600 may proceed to step 610, where the DPTT may be determined using an alternative algorithm. Illustrative alternative algorithms are depicted in FIGS. 7 and 8, and will be described in greater detail later herein in connection with those figures. Steps 608 and 610 may be performed by microprocessor 48 (FIG. 2) and/or processor 312 (FIG. 3) of a CNIBP monitoring or pulse oximetry system.

Advantageously, the approach depicted in FIG. 6 allows the DPTT to be determined more accurately in cases when the default maximum correlation algorithm performed in steps 602 and 604 yields results that are relatively unreliable. In those cases, the DPTT may be determined using an alternative algorithm in step 610 that may be less susceptible to the errors that caused the relatively unreliable results in the original maximum correlation algorithm. In this way, the accuracy of the resulting blood pressure measurements may be improved.

In practice, one or more steps shown in process 600 may be combined with other steps, performed in any suitable order, performed in parallel (e.g., simultaneously or substantially simultaneously), or removed.

FIG. 7 shows a first illustrative alternative algorithm 700 for determining a DPTT measurement from PPG signals in accordance with an embodiment. Alternative algorithm 700 may be used as part of step 610 of process 600 (FIG. 6). At step 702, the second derivative of each filtered PPG signal may be computed. The second derivatives may be computed by microprocessor 48 (FIG. 2) and/or processor 312 (FIG. 3) of a CNIBP monitoring or pulse oximetry system. In alternative embodiments, the first derivative of each filtered PPG signal may be computed instead, or the filtered PPG signals themselves may be used in alternative algorithm 700 without any derivative operations.

At step 704, a set of peaks may be identified in each of the second-derivative waveforms generated at step 702. The peaks may be identified by microprocessor 48 (FIG. 2) and/or processor 312 (FIG. 3) of a CNIBP monitoring or pulse oximetry system, and may be identified in any suitable manner. For example, a derivative of each second-derivative waveform may be generated and times at which the value of the generated derivative equals zero may be recorded. As another example, peaks may be identified in the second-derivative waveform directly without any further derivative operations. In an embodiment, only peaks above a certain height, wider than a certain width, or both are identified. Such height and width requirements may advantageously filter out false peaks generated by noise or otherwise not corresponding to beats of the patient's heart.

At step 706, the average time between corresponding peaks identified at step 704 may be determined. The average time may be determined by microprocessor 48 (FIG. 2) and/or processor 312 (FIG. 3) of a CNIBP monitoring or pulse oximetry system, and may be determined in any suitable manner. For instance, each pair of corresponding peaks may be iterated through in turn, and the time between the two peaks may be added to a running total. When all peaks have been iterated through, the total may be divided by the number of pairs examined, to yield an average time. That average time may then be used as an alternative DPTT measurement in computing a patient's blood pressure.

In practice, one or more steps shown in process 700 may be combined with other steps, performed in any suitable order, performed in parallel (e.g., simultaneously or substantially simultaneously), or removed.

FIG. 8 shows a second illustrative alternative algorithm 800 for determining a DPTT measurement from PPG signals in accordance with an embodiment. Alternative algorithm 800 may be used as part of step 610 of process 600 (FIG. 6). At step 802, the second derivative of each filtered PPG signal may be computed. The second derivatives may be computed by microprocessor 48 (FIG. 2) and/or processor 312 (FIG. 3) of a CNIBP monitoring or pulse oximetry system. In alternative embodiments, the filtered PPG signals themselves may be used in alternative algorithm 800 without any derivative operations.

At step 804, a maximum correlation algorithm may be performed on the second-derivative waveforms generated at step 802. The maximum correlation algorithm may be performed by microprocessor 48 (FIG. 2) and/or processor 312 (FIG. 3) of a CNIBP monitoring or pulse oximetry system. The maximum correlation algorithm may be substantially similar to the algorithm performed at step 604 of process 600 (FIG. 6), with the inputs to the algorithm now being the second derivatives of the PPG signals rather than the first derivatives.

At step 806, the DPTT may be determined based on the results of the maximum correlation algorithm. The determination may be performed by microprocessor 48 (FIG. 2) and/or processor 312 (FIG. 3) of a CNIBP monitoring or pulse oximetry system, and may be substantially similar to that of step 608 of process 600 (FIG. 6). In particular, the DPTT may be set to be the amount of time shift identified at step 804 as maximizing the correlation between the two second-derivative waveforms, according to any suitable correlation metric.

In practice, one or more steps shown in process 800 may be combined with other steps, performed in any suitable order, performed in parallel (e.g., simultaneously or substantially simultaneously), or removed.

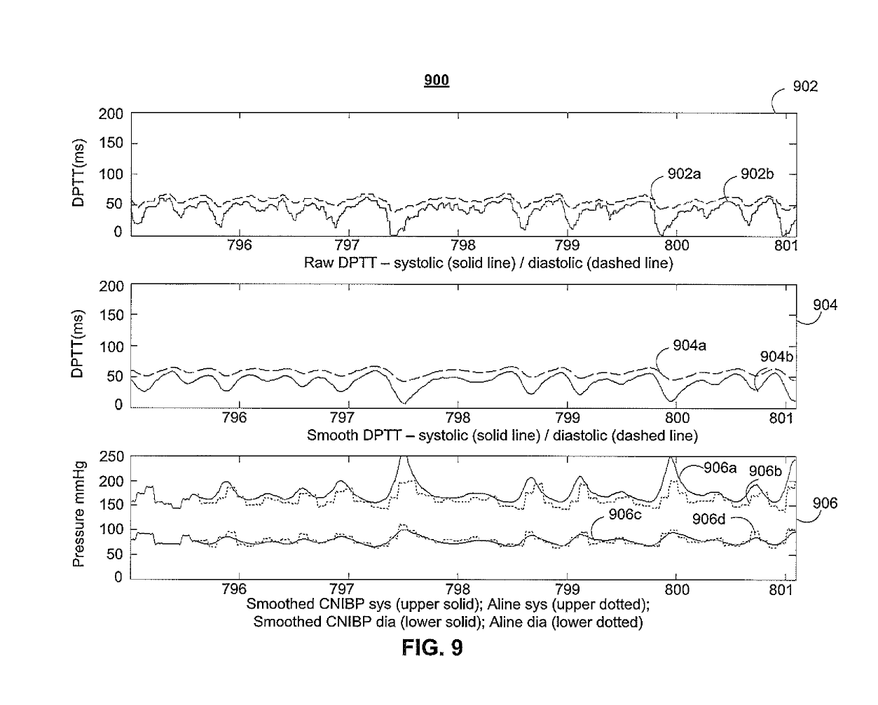

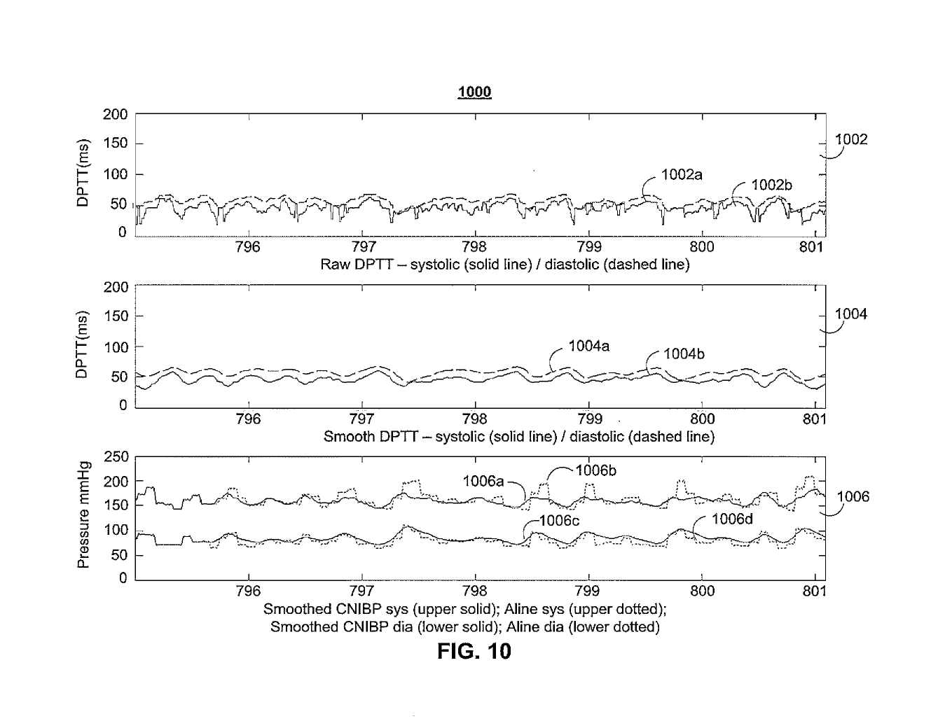

FIG. 9 shows a set of illustrative waveforms depicting blood pressure determination using a DPTT measurement computed with a default maximum correlation algorithm in accordance with an embodiment. Both FIG. 9 and FIG. 10 depict waveforms for “diastolic” measurements, corresponding to expansion of the heart chambers, and “systolic” measurements, corresponding to contraction of the heart chambers. Diastolic and systolic blood pressure measurements may be determined in any suitable manner. For example, in FIGS. 9 and 10, it is assumed that two different sets of DPTT measurements are taken, one diastolic and one systolic. In this case, each set of DPTT measurements may be used to compute a corresponding set of blood pressure measurements (e.g., by using equation (1) with two sets of values for T). As another example, a single set of DPTT measurements may be used to compute separate diastolic and systolic blood pressure measurements. In this case, equation (1) (or another suitable equation relating DPTT to blood pressure) may be used with one set of a and b values to determine diastolic blood pressure and another set of a and b values to determine systolic blood pressure. It will be understood that the methods depicted in FIGS. 5-8 may be used with diastolic measurements, systolic measurements, or any suitable combination thereof.

In practice, certain sets of measurements may be relatively well-adapted for computing diastolic DPTT and blood pressure values, while other sets of measurements may be relatively well-adapted for computing systolic DPTT and blood pressure values. For example, diastolic DPTT and blood pressure values may be computed relatively accurately using the first derivative of incoming PPG signals (as compared to, e.g., the raw PPG signals or their second derivatives). Similarly, systolic DPTT and blood pressure values may be computed relatively accurately using the second derivative of incoming PPG signals (as compared to, e.g., the raw PPG signals or their first derivatives). It will be understood, however, that any suitable set of PPG measurements may be used to compute diastolic and systolic DPTT and blood pressure values, and the disclosure is not limited in this respect.

Illustrative graph 902 includes an example waveform 902a representing a patient's diastolic DPTT as it varies with time, computed using the default maximum correlation algorithm based on the first derivative of incoming PPG signals. Example waveform 902b represents the patient's systolic DPTT as it varies with time, computed using the default maximum correlation algorithm based on the second derivative of the PPG signals. In this illustrative example, the DPTT measurements include zero crossings that adversely affect the accuracy of the blood pressure measurement. Thus, diastolic waveform 902a exhibits relatively substantial deviation from systolic waveform 902b.

Illustrative graph 904 includes example waveforms 904a and 904b, representing smoothed versions of the patient's diastolic DPTT waveform 902a and systolic waveform 902b, respectively. Smoothed waveforms 904a and 904b may be generated with any suitable technique, such as low-pass filtering. Again, smoothed diastolic waveform 904a exhibits relatively substantial deviation from smoothed systolic waveform 904b.

Illustrative graph 906 depicts blood pressure measurements that may be generated using the DPTT data of graph 904. Waveform 906a may represent the systolic blood pressure estimate, while waveform 906c may represent the diastolic blood pressure estimate. Additionally included in graph 906 is waveform 906b, which may represent the patient's a-line systolic blood pressure measurement of the patient, and waveform 906d, which may represent the patient's a-line diastolic blood pressure measurement. Because a-line measurements are usually generated using a device that is directly inserted into a patient's blood vessel, they are considered relatively accurate and are thus often used to gauge the accuracy of non-invasive blood pressure measurements. Here, the non-invasive blood pressure measurements represented by systolic waveform 906a show noticeable deviations from a-line systolic waveform 906b due to the incorporation of zero crossings into the original DPTT measurements. On the other hand, in this example diastolic waveform 906c seems relatively well-aligned with a-line diastolic waveform 906d, reflecting the fact that second-derivative PPG measurements may be less susceptible to noise than first-derivative PPG measurements.

FIG. 10 shows a set of illustrative waveforms depicting blood pressure determination using a DPTT measurement computed at least partially with an alternative algorithm in accordance with an embodiment. Illustrative graph 1002 includes an example waveform 1002a representing a patient's diastolic DPTT as it varies with time. Example waveform 1002b represents the patient's systolic DPTT as it varies with time. In this illustrative example, the DPTT measurements of waveforms 1002a and 1002b have been advantageously computed using an approach such as that depicted in FIG. 6, which detects relatively low-confidence correlation results and computes the DPTT using an alternative algorithm when such a detection occurs. Thus, diastolic waveform 1002a and systolic waveform 1002b match more closely than corresponding respective waveforms 902a and 902b (FIG. 9).

Illustrative graph 1004 includes example waveforms 1004a and 1004b, representing smoothed versions of the patient's diastolic DPTT waveform 1002a and systolic waveform 1002b, respectively. Smoothed waveforms 1004a and 1004b may be generated with any suitable technique, such as low-pass filtering. Again, smoothed diastolic waveform 1004a and smoothed systolic waveform 1004b match more closely than corresponding respective waveforms 904a and 904b (FIG. 9).

Illustrative graph 1006 depicts blood pressure measurements that may be generated using the DPTT data of graph 1004. Waveform 1006a may represent the systolic blood pressure estimate, while waveform 1006c may represent the diastolic blood pressure estimate. Additionally included in graph 1006 is waveform 1006b, which may represent the patient's a-line systolic blood pressure measurement of the patient, and waveform 1006d, which may represent the patient's a-line diastolic blood pressure measurement. Because a-line measurements are usually generated using a device that is directly inserted into a patient's blood vessel, they are considered relatively accurate and are thus often used to gauge the accuracy of non-invasive blood pressure measurements. Here, the non-invasive blood pressure measurements represented by systolic waveform 1006a match more closely than corresponding respective waveform 906a (FIG. 9). Diastolic waveform 1006c appears approximately as well-aligned as corresponding waveform 906c (FIG. 9), again reflecting the fact that second-derivative PPG measurements may be less susceptible to noise than first-derivative PPG measurements.

The foregoing is merely illustrative of the principles of this disclosure and various modifications can be made by those skilled in the art without departing from the scope and spirit of the disclosure. The above described embodiments are presented for purposes of illustration and not of limitation. The present disclosure also can take many forms other than those explicitly described herein. For example, steps 602 and 604 (FIG. 6) may be replaced with steps representing any suitable algorithm that can be used to compute a DPTT value. As another example, two or more algorithms may be performed in parallel to compute two or more DPTT measurements, a confidence measure may be determined for the results of each of those algorithms, and the highest-confidence DPTT measurement may be used to compute the blood pressure measurement. As another example, the two more DPTT measurements may be averaged together based on fixed weights or variable weights (e.g., selected based on the determined confidence measures) to obtain a final DPTT measurement for use in computing the blood pressure measurement. Other variations are possible. Accordingly, it is emphasized that the disclosure is not limited to the explicitly disclosed methods, systems, and apparatuses, but is intended to include variations to and modifications thereof which are within the spirit of the following claims.

What is claimed is:

1. A method for processing first and second photoplethysmograph (PPG) signals to determine a differential pulse transit time (DPTT), the method comprising:
detecting the first and second PPG signals;
performing a DPTT algorithm based at least in part on the first and second PPG signals to obtain a DPTT;
determining a confidence measure associated with the DPTT obtained using the DPTT algorithm;
determining whether to use the DPTT based at least in part on the confidence measure; and
when it is determined not to use the DPTT, performing an alternative DPTT algorithm based at least in part on the first and second PPG signals to obtain the DPTT.

2. The method of claim 1 further comprising determining a blood pressure measurement based on the DPTT.

3. The method of claim 1 wherein performing the DPTT algorithm comprises:
computing a first derivative of each of the first and second PPG signals; and
performing a maximum correlation algorithm based on the computed first derivatives.

4. The method of claim 3 wherein the confidence measure is indicative of how far the first derivative of the first PPG signal must be shifted in time relative to the first derivative of the second PPG signal in order to maximize correlation between the first derivatives.

5. The method of claim 4 wherein the DPTT obtained using the DPTT algorithm is used when the confidence measure is in a range containing numbers greater than zero and wherein the DPTT obtained using the DPTT algorithm is not used when the confidence measure is in a range containing numbers less than zero.

6. The method of claim 3 wherein the confidence measure is indicative of how highly correlated the first derivative of the first PPG signal is with the first derivative of the second PPG signal after one of the first derivatives is shifted in time relative to the other of the first derivatives.

7. The method of claim 6 wherein the DPTT obtained using the DPTT algorithm is used when the confidence measure is greater than a threshold.

8. The method of claim 1 wherein performing the alternative DPTT algorithm comprises computing a second derivative of each of the first and second PPG signals.

9. The method of claim 8 wherein performing the alternative DPTT algorithm further comprises:
identifying a first set of peaks in the second derivative of the first PPG signal;
identifying a second set of peaks in the second derivative of the second PPG signal;
determining an average time difference between respective peaks in the first and second set of peaks; and
outputting the average time difference as the DPTT.

10. The method of claim 1 wherein performing the alternative DPTT algorithm comprises performing a maximum correlation algorithm based at least in part on the first and second PPG signals.

11. A system for processing first and second photoplethysmograph (PPG) signals to determine a differential pulse transit time (DPTT), the system comprising:
at least one sensor capable of generating the first and second PPG signals; and
a processor capable of
receiving the first and second PPG signals;
performing a DPTT algorithm based at least in part on the first and second PPG signals to obtain a DPTT;
determining a confidence measure associated with the DPTT obtained using the DPTT algorithm;
determining whether to use the DPTT based at least in part on the confidence measure; and
when it is determined not to use the DPTT, performing an alternative DPTT algorithm based at least in part on the first and second PPG signals to obtain the DPTT.

12. The system of claim 11 wherein the processor is further capable of determining a blood pressure measurement based on the DPTT.

13. The system of claim 11 wherein performing the DPTT algorithm comprises:
computing a first derivative of each of the first and second PPG signals; and
performing a maximum correlation algorithm based on the computed first derivatives.

14. The system of claim 13 wherein the confidence measure is indicative of how far the first derivative of the first PPG signal must be shifted in time relative to the first derivative of the second PPG signal in order to maximize correlation between the first derivatives.

15. The system of claim 14 wherein the DPTT obtained using the DPTT algorithm is used when the confidence measure is in a range containing numbers greater than zero and wherein the DPTT obtained using the DPTT algorithm is not used when the confidence measure is in a range containing numbers less than zero.

16. The system of claim 13 wherein the confidence measure is indicative of how highly correlated the first derivative of the first PPG signal is with the first derivative of the second PPG signal after one of the first derivatives is shifted in time relative to the other of the first derivatives.

17. The system of claim 16 wherein the DPTT obtained using the DPTT algorithm is used when the confidence measure is greater than a threshold.

18. The system of claim 11 wherein performing the alternative DPTT algorithm comprises computing a second derivative of each of the first and second PPG signals.

19. The system of claim 18 wherein performing the alternative DPTT algorithm comprises:
identifying a first set of peaks in the second derivative of the first PPG signal;
identifying a second set of peaks in the second derivative of the second PPG signal;
determining an average time difference between respective peaks in the first and second set of peaks; and
outputting the average time difference as the DPTT.

20. The system of claim 11 wherein performing the alternative DPTT algorithm comprises performing a second maximum correlation algorithm based on the first and second PPG signals.
